(19) United States
(12) Patent Application Publication
(10) Pub. No.: US 2019/0380886 A1
(43) Pub. Date: Dec. 19, 2019

(54) Clothing for Enuresis

(71) Applicant: Craig Hammond, Millcreek, UT (US)

(72) Inventor: Craig Hammond, Millcreek, UT (US)

(21) Appl. No.: 16/441,705

(22) Filed: Jun. 14, 2019

Related U.S. Application Data
(60) Provisional application No. 62/685,782, filed on Jun. 15, 2018.

(51) Int. Cl. A61F 13/49; A61F 13/42; A61F 13/531

(52) U.S. Cl. CPC A61F 13/49006; A61F 13/42; A61F 2013/530007; A61F 2013/425; A61F 13/531

(57) ABSTRACT

An article of clothing includes an outer portion and an inner portion with relatively thin profiles to imitate typical pajama pants and underwear. The inner portion retains liquids in the crotch region and prevents leaks, despite its relatively thin profile. The inner portion also includes leg holes constructed to create liquid-retaining barriers around the user's legs by cinching around the outer regions of the user's legs. This enables the material forming the portions of the leg holes around the inner regions of the user's legs to remain relatively flat, leading to increased comfort and a reduced profile around the crotch.

### RELATED APPLICATIONS

This application claims the benefit of and priority to U.S. Provisional Patent Application No. 62/685,782, filed Jun. 15, 2018, and entitled “Clothing for Enuresis.” This application relates to U.S. Design patent application Ser. No. 29/653,578, filed Jun. 15, 2018, and entitled “Article of Clothing for Enuresis.” The contents of these applications are incorporated by reference herein in their entireties for all purposes.

### BACKGROUND

As many parents and caretakers know, potty-training can be challenging for children, particularly at night time. During this transition period, the use of diapers tends to encourage urination, while the use of normal underwear often results in soiled clothing and bedsheets. Furthermore, the costs of diapers and/or the additional laundry work can quickly climb to a significant sum and cause negative environmental impacts.

### SUMMARY

According to some embodiments of the present disclosure, an article of clothing includes an outer portion and an inner portion. The outer portion is designed to imitate typical pajama pants. The inner portion is permanently attached to the outer portion and is relatively thin, such that the article of clothing has a profile substantially similar to a profile of typical pajama pants, as opposed to a profile created by a diaper. This helps users (especially young children) in their potty-training efforts, as using a diaper tends to encourage children to urinate. At the same time, the inner portion is designed to retain liquid in the crotch region and prevent leaks, despite its relatively thin profile. In some embodiments, the inner portion includes leg holes constructed to create liquid-retaining barriers around the user's legs by cinching around the outer regions of the user's legs. This enables the material forming the portions of the leg holes around the inner regions of the user's legs to remain relatively smooth, leading to increased comfort and a reduced profile around the crotch. According to some embodiments, similar articles of clothing may be provided for users that are not in a potty-training phase (for example, incontinent individuals).

In an exemplary embodiment of the present disclosure, clothing bottoms are configured to be worn on a lower portion of a user. The clothing bottoms include an exterior portion and an interion portion. The exterior portion includes a crotch region, a first leg region coupled to the crotch region and configured to receive a first leg of the user, and a second leg region coupled to the crotch region and configured to receive a second leg of the user. The interior portion is coupled to the exterior portion and disposed within the crotch region of the exterior portion. The interior portion is configured to receive a crotch portion of the user therein and retain liquids therein when the clothing bottoms is worn by the user. The clothing bottoms are washable and reusable.

The clothing bottoms as described herein above, wherein the interior portion includes at least one absorbent material configured to retain a multiple of its weight in liquid.

The clothing bottoms as described herein above, wherein the interior portion is configured to retain fluids while providing a slightly wet sensation.

The clothing bottoms as described herein above, wherein the interior portion includes a blend of absorbent materials.

The clothing bottoms as described herein above, wherein the interior portion further includes polyester.

The clothing bottoms as described herein above, wherein the interior portion includes a first layer and a second layer. The first layer is configured to contact the user, and the first layer includes an absorbent material. The second layer is disposed between the first layer and the exterior portion, and the second layer includes an absorbent material.

The clothing bottoms as described herein above, wherein the first layer includes at least one of absorbent bamboo fabric and wicking fabric.

The clothing bottoms as described herein above, wherein the interior portion further includes a third layer disposed between the second layer and the exterior portion.

The clothing bottoms as described herein above, wherein the third layer includes an impermeable material.

The clothing bottoms as described herein above, wherein the interior portion further includes a first leg hole perimeter and a second leg hole perimeter. The first leg hole perimeter defines a first leg hole configured to receive the first leg of the user, and the first leg hole perimeter is configured to provide a first liquid-retaining barrier around the first leg of the user. The second leg hole perimeter defines a second leg hole configured to receive the second leg of the user, and the second leg hole perimeter is configured to provide a second liquid-retaining barrier around the second leg of the user.

The clothing bottoms as described herein above, wherein the first leg hole perimeter is configured to continuously circumscribe the first leg of the user, and the second leg hole perimeter is configured to continuously circumscribe the second leg of the user.

The clothing bottoms as described herein above, wherein the first leg hole perimeter includes a first finishing seam and the second leg hole perimeter includes a second finishing seam.

The clothing bottoms as described herein above, wherein the first finishing seam and the second finishing seam each include around 70% nylon and around 30% spandex.

The clothing bottoms as described herein above, wherein the clothing bottoms lacks athletic fabric or wicking fabric configured to contact the user to facilitate providing the user with a slightly wet sensation.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a front perspective view of clothing bottoms, according to embodiments of the present disclosure.

FIG. 2 shows a back view of the clothing bottoms of FIG. 1.

FIG. 3 shows a front view of the clothing bottoms of FIG. 1.

FIG. 4 shows a right side view of the clothing bottoms of FIG. 1.

FIG. 5 shows a left side view of the clothing bottoms of FIG. 1.

FIG. 6 shows a top view of the clothing bottoms of FIG. 1.

FIG. 7 shows a bottom view of the clothing bottoms of FIG. 1.

FIG. 8 shows a front view of the clothing bottoms of FIG. 1, with the clothing bottoms turned inside out.

FIG. 9 shows a back view of the clothing bottoms of FIG. 1, with the clothing bottoms turned inside out.

FIG. 10 shows a right side view of the clothing bottoms of FIG. 1, with the clothing bottoms turned inside out.

FIG. 11 shows a left side view of the clothing bottoms of FIG. 1, with the clothing bottoms turned inside out.

FIG. 12 shows a top view of the clothing bottoms of FIG. 1, with the clothing bottoms turned inside out.

FIG. 13 shows a bottom view of the clothing bottoms of FIG. 1, with the clothing bottoms turned inside out.

FIG. 14 shows a side sectional view of a crotch region of the clothing bottoms of FIG. 1.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

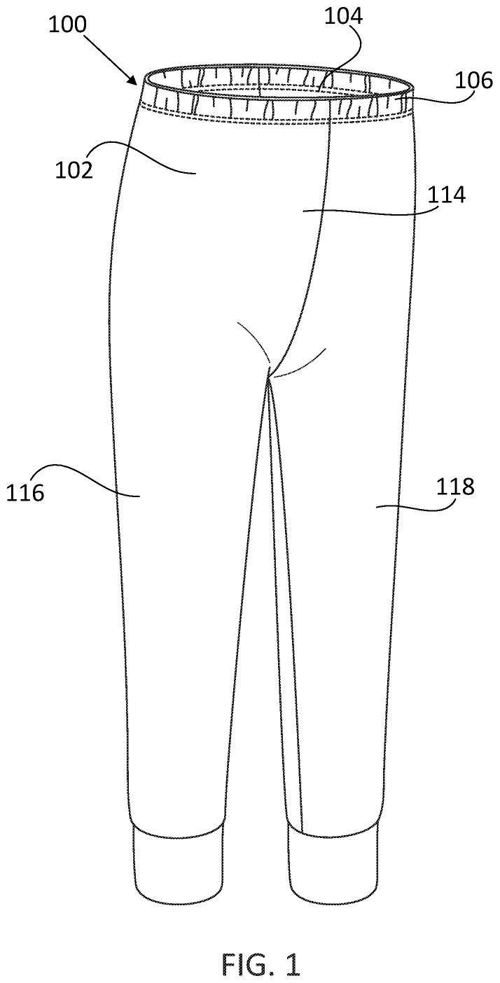

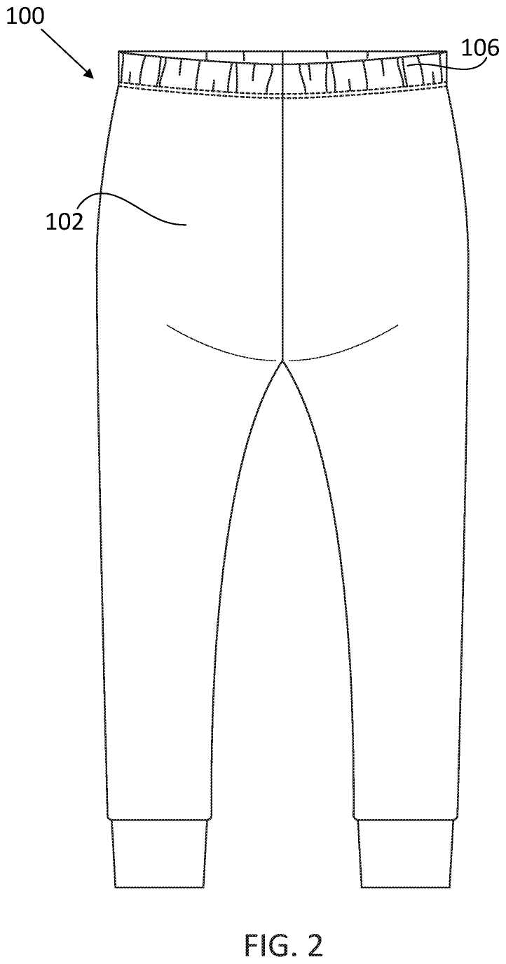

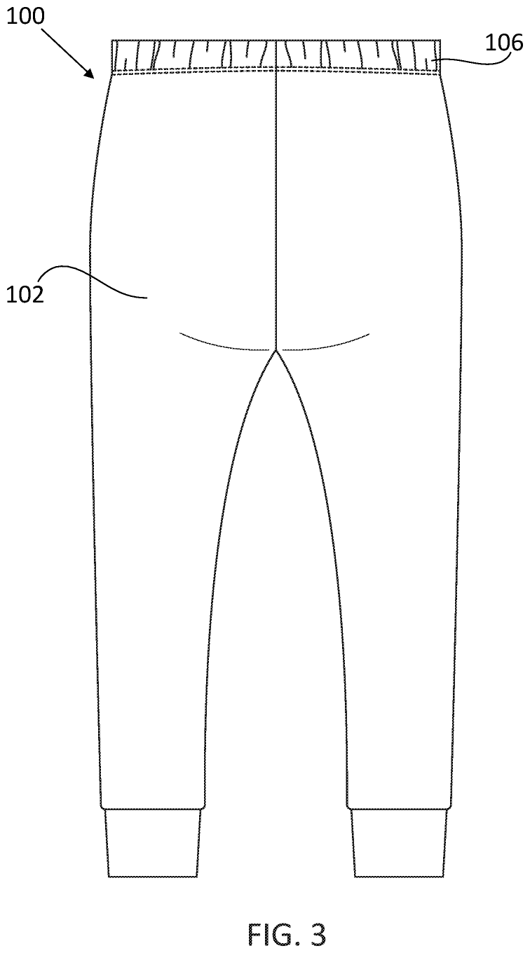

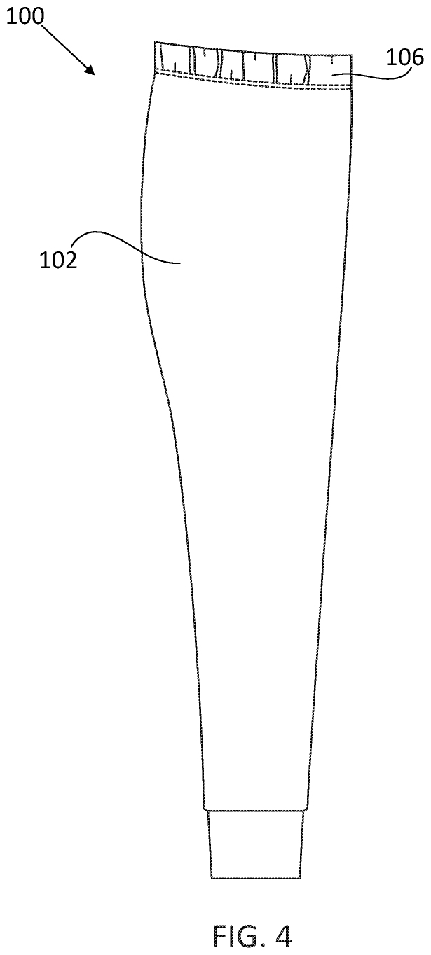

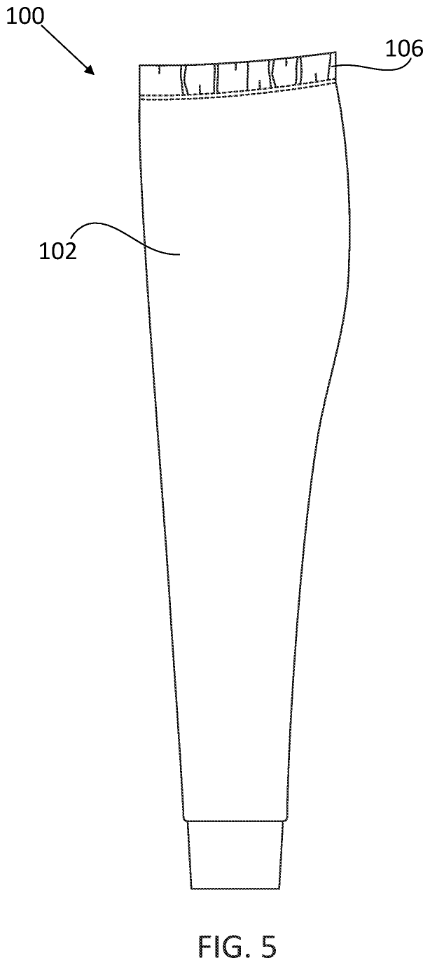

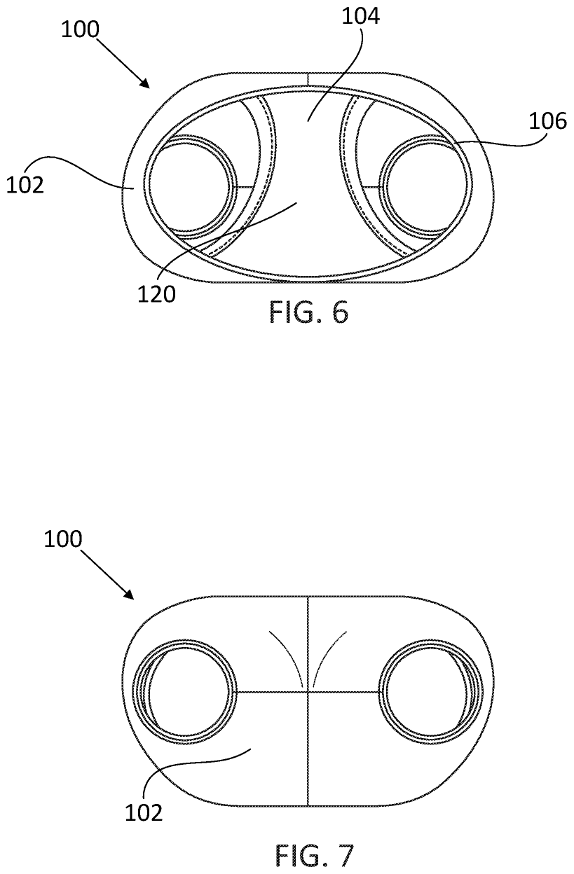

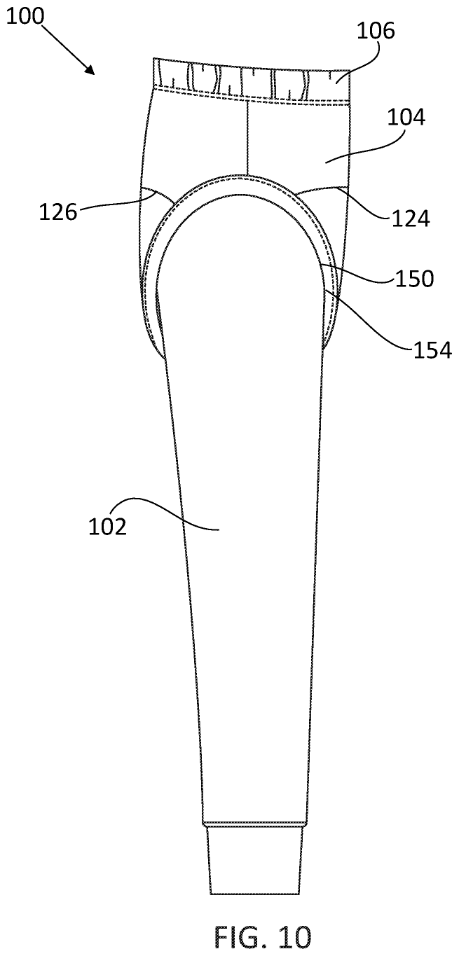

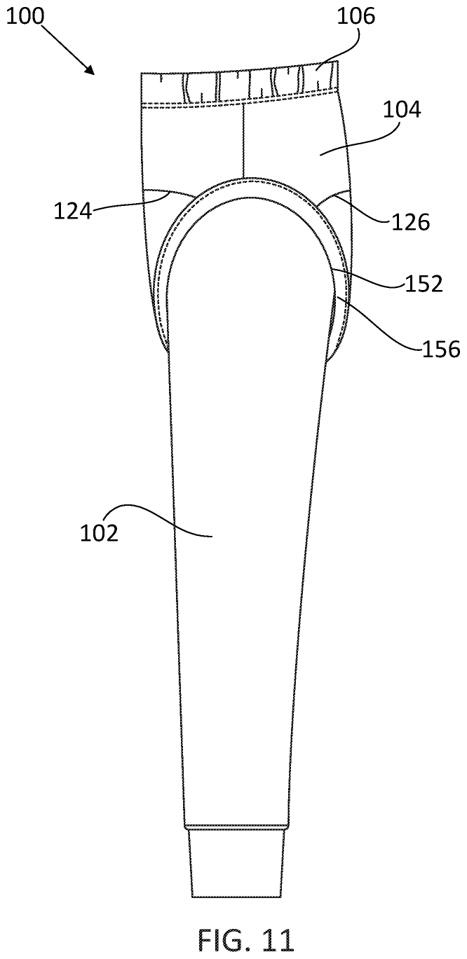

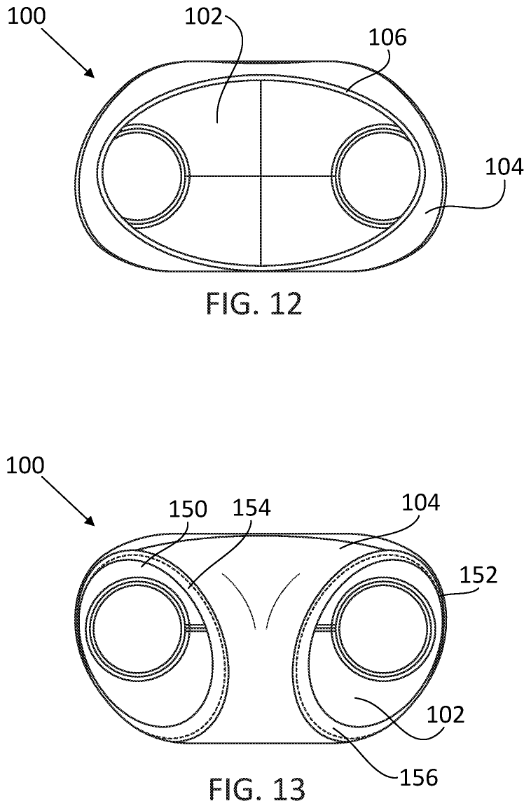

As shown in FIGS. 1-13, in some embodiments clothing bottoms 100 includes an exterior portion 102 and an interior portion 104 that are permanently attached, for example, through stitching in the waistband 106. As discussed below in more detail, the clothing bottoms 100 has a profile that substantially corresponds to a profile of a typical pair of pajama bottoms (and includes, for example, a crotch region 114, a first leg region 116, and a second leg region 118) and underwear, as opposed to the larger and bulkier profile that results from the use of diapers, pull-ups, or similar products designed to prevent leaking fluids from urination. As a result, the clothing bottoms in these embodiments provides a realistic feel that encourages children to continue to strive for dry nights.

For example, in some embodiments a thickness of the clothing bottoms 100, measured from the front facing surface of the exterior portion 102 at the crotch region 114 to the rear facing surface of the interior portion 104 approximates the thickness of the average pajama pants and underwear. This feature can also be measured as a thickness from the interior facing surface of the interior portion 104 (i.e., the surface that would contact the wearer) to the front facing surface of the exterior portion 102. Diapers and pull-ups, in contrast, are often significantly thicker and bulkier due to the location and composition of the materials required to absorb and maintain bodily waste. This difference can be perceived visually to the average user, including the average potty-training child.

In some embodiments, the profile of the exterior portion 102 of the clothing bottoms 100 is substantially identical to that of an average pair of pajamas bottoms. Thus, the difference in the overall profile between the clothing bottoms 100 and the profile created by a diaper (or pull-up or similar article) in combination with pajama pants is largely due to the difference in the profile of the interior portion 104 and the diaper.

The distinct profile of a clothing bottoms 100 (e.g., the interior portion 104) relative to a diaper (or pull-up or similar article) becomes even more pronounced once those articles become soiled. In such situations, the materials in the diaper or pull up expand as they absorb the waste materials, enlarging the profile and increasing the bulky appearance. The materials of the interior portion 104, as discussed below in more detail, retain the waste materials with a much smaller expansion in profile and perceived bulkiness.

Despite that relatively thin profile, the interior portion 104 is constructed to prevent leaking fluids from urination. For example, in some embodiments the interior portion 104 is formed of absorbent materials that can hold a multiple of their weight in liquid (for example, over ten times their weight in liquid). In addition, in some embodiments, the boundaries of the interior portion 104 are configured to form liquid retaining boundaries with the user's skin, for example, through the use of elastic material designed to create those liquid retaining boundaries without overly clamping on the user. In some embodiments, the interior portion 104 can retain fluids while still providing a slightly wet sensation, so that the user knows that urination has begun and can respond accordingly.

In particular, the interior portion 104 is formed from several materials designed to absorb, wick, and contain urine. For example, those materials may include bamboo/cotton blends, with some polyester added for stretch. Additional materials may include a wicking-type fabric that can help to decrease the sensation of feeling wet in certain designs (for older kids and incontinent adults), microfiber fleece material for maximum absorbency, and/or PUL, TPU, or a similar impermeable fabric to keep the liquid contained.

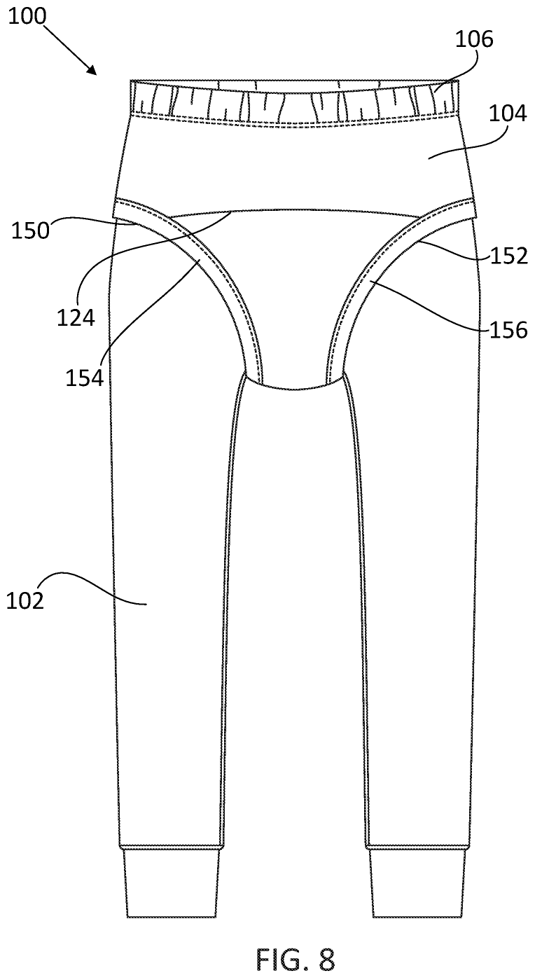

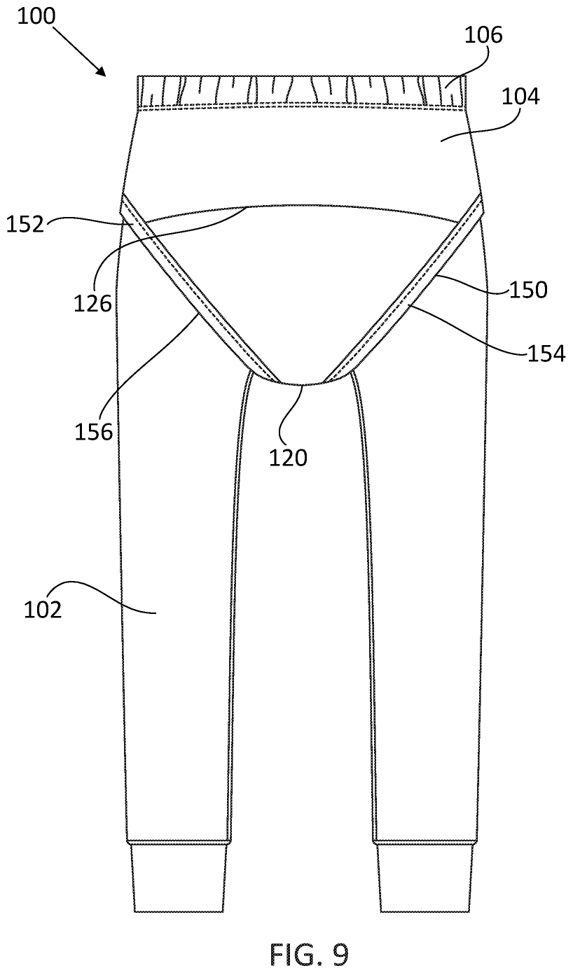

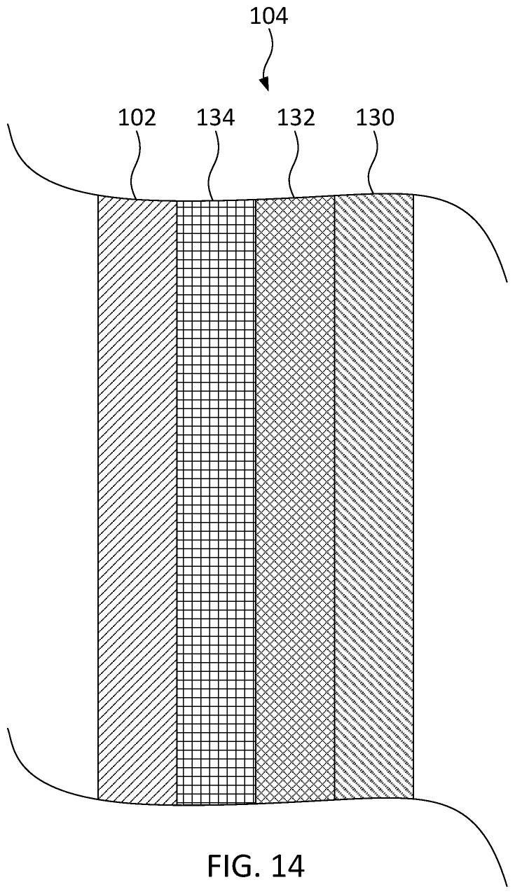

In some embodiments, the interior portion 104 is formed from several different layers. For example and referring specifically to FIG. 14, a first or an interior-most layer 130 (i.e., the layer that contacts the user) is formed from absorbent bamboo or wicking fabrics. In some embodiments, this interior-most layer 130 extends from the waistband 106 at the top of the clothing bottoms 100 down to the bottoms 120 of the interior portion 104. In some embodiments, this interior-most layer 130 entirely covers the groin and buttocks with two leg holes, just like typical underwear. The next layer out, or a second layer 132, is formed from one or more secondary absorbent portions that are sewn extending from approximately the top of the pubic bone in the front to approximately the upper part of buttocks in the back. In FIGS. 8-9, the upper limits of the second layer 132 are identified with seams 124 and 126. The second layer 132 provides secondary protection (and tertiary protection in some embodiments) to maximize absorption and decrease spillage. A third layer 134 is formed from an impermeable fabric that covers the entirety of the inside/liner from the waist to the thigh and to the leg holes, similar to the profile of the interior-most layer.

As also shown in, e.g., FIG. 8, the interior portion 104 includes first and second leg holes 150, 152 that are defined by first and second leg hole perimeters 154, 156. In some embodiments and as illustrated, the leg hole perimeters 154, 156 are provide as or include finishing seams 154, 156. The finishing seams 154, 156 provide additional comfort to the user and help ‘seal’ or form liquid-retaining barriers that prevent liquid from leaking through creases or space in the leg holes. In some embodiments, these leg holes are formed without using an elastic band. In other embodiments, the interior portion 104 includes an elastic band located over only the outer regions of the user's legs, so that any cinching from the elastic occurs only over those outer regions.

In some embodiments the material of the seams 154, 156 is a blend of around 70% nylon and around 30% spandex (around being understood as within ±5%). This material provides a stretchiness or elasticity to the leg holes 150 that can constrict over the user's legs and prevent fluids from flowing down the user's legs. In some embodiments, the finishing seams 154, 156 are constructed with the portion of the material on the outside of the leg providing the majority of the stretch and elasticity, in comparison to the material on the inside of the leg though in the crotch or groin region. In some embodiments, only the portions of the material on the outside of the leg provide the stretch and elasticity needed to create the liquid barriers.

In some embodiments, the elasticity is centralized to the outer region of the leg because there is more fabric around the waist/hip for the elastic to tighten or cinch. Rather than using elastic that could be too tight and uniformly pull the fabric together to clamp down on the user's legs, which could lead to pinching, discomfort or even injury, the interior portion 104 places an “excess” or quantity of fabric in the upper portion of the interior portion 104 to pull or cinch the excess fabric tighter on the upper outside portion of the leg. In other embodiments, the materials around the outer region of the user's legs are different than the materials around the inner region of the user's legs.

Stated differently, through the use of, e.g., different materials or different amounts of material, the cinching of the interior portion 104 to create those liquid retaining barriers can be isolated to the outer regions of the user's legs, enabling the material around the inner regions of the user's legs to remain relatively smooth and un-cinched. In this manner, the interior portion 104 provides security from leaking while minimizing or eliminating any discomfort or pinching that typical elastic bands often cause. Considering the sensitivity of the body parts in the inner groin, these embodiments reduce any pinching, unnecessary seams, or discomfort in the groin region.

In these embodiments, the interior portion 104 maintains urine or liquids specifically in the groin region. The materials around the leg holes create the fluid-retaining barriers, preventing urine from running down the lower or upper leg in a way that overcomes problems with traditional elastic solutions that cinch around the inner portions of the user's legs. Moreover, the interior portion in these embodiments keeps the cuff from acting as a seal.

In this regard, the interior portion 104 provides improvements over typical diapers and other approaches. Diapers, for example, are made to fasten around the child (e.g., by wrapping around the hips). As a result, those diapers do not employ leg holes that continuously circumscribe the user's legs. Pull-ups are designed to part on the side, splitting down into the portion of the pull-up surrounding the user's legs. Thus, those pull-ups also do not employ leg holes that continuously circumscribe the user's legs. Furthermore, diapers and pull-ups often concentrate elasticity around the inner regions of the user's legs, partly in view of their constructions and partly to retain the bulky, expanded materials that absorb the bodily fluids. In some embodiments, the interior portion 104, in contrast, concentrates the elasticity along the upper regions of the user's legs (in some embodiments, exclusively along the upper regions of the user's legs). As a result, the inner regions of the user's legs contact a relatively smooth surface, thereby forming a moisture-barrier without the discomfort created by cinched material in that area.

The article of clothing 100 may be reusable. More specifically, after being soiled the article of clothing 100 can be washed as part of a normal washing load. In some embodiments, the article of clothing 100 may be washed after being soiled and reused various times (for example, over 300 times) before its absorbency is significantly reduced. The interior-most layer of the interior portion 104 can be made of a soft material to encourage comfort and use. The waistband also serves to retain fluids.

In some embodiments, the interior portion 104 is configured to operate primarily by retaining the fluids in contact with the user, rather than wicking the moisture away from the user. In some embodiments, the article of clothing 100 lacks athletic fabric or wicking fabric in contact with the user's skin to facilitate providing the user with a slightly wet sensation. In other embodiments, the interior portion 104 is configured to wick moisture away from the user to promote drier skin.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. Clothing bottoms configured to be worn on a lower portion of a user, the clothing bottoms comprising:
an exterior portion comprising:
a crotch region;
a first leg region coupled to the crotch region and configured to receive a first leg of the user;
a second leg region coupled to the crotch region and configured to receive a second leg of the user; and
an interior portion coupled to the exterior portion and disposed within the crotch region of the exterior portion, the interior portion configured to receive a crotch portion of the user therein and retain liquids therein when the clothing bottoms is worn by the user;
wherein the clothing bottoms are washable and reusable.

2. The clothing bottoms of claim 1, wherein the interior portion comprises at least one absorbent material configured to retain a multiple of its weight in liquid.

3. The clothing bottoms of claim 1, wherein the interior portion is configured to retain fluids while providing a slightly wet sensation.

4. The clothing bottoms of claim 1, wherein the interior portion comprises a blend of absorbent materials.

5. The clothing bottoms of claim 4, wherein the interior portion further comprises polyester.

6. The clothing bottoms of claim 1, wherein the interior portion comprises:
a first layer configured to contact the user, the first layer comprising an absorbent material; and
a second layer disposed between the first layer and the exterior portion, the second layer comprising an absorbent material.

7. The clothing bottoms of claim 6, wherein the first layer comprises at least one of absorbent bamboo fabric and wicking fabric.

8. The clothing bottoms of claim 6, wherein the interior portion further comprises a third layer disposed between the second layer and the exterior portion.

9. The clothing bottoms of claim 8, wherein the third layer comprises an impermeable material.

10. The clothing bottoms of claim 1, wherein the interior portion further comprises:
a first leg hole perimeter defining a first leg hole configured to receive the first leg of the user; and the first leg hole perimeter configured to provide a first liquid-retaining barrier around the first leg of the user; and
a second leg hole perimeter defining a second leg hole configured to receive the second leg of the user; and the second leg hole perimeter configured to provide a second liquid-retaining barrier around the second leg of the user.

11. The clothing bottoms of claim 10, wherein the first leg hole perimeter is configured to continuously circumscribe the first leg of the user, and the second leg hole perimeter is configured to continuously circumscribe the second leg of the user.

12. The clothing bottoms of claim 10, wherein the first leg hole perimeter comprises a first finishing seam and the second leg hole perimeter comprises a second finishing seam.

13. The clothing bottoms of claim 12, wherein the first finishing seam and the second finishing seam each comprise around 70% nylon and around 30% spandex.

14. The clothing bottoms of claim 1, wherein the clothing bottoms lacks athletic fabric or wicking fabric configured to contact the user to facilitate providing the user with a slightly wet sensation.
